(19) United States
(12) Patent Application Publication
(10) Pub. No.: US 2014/0343575 A1
(43) Pub. Date: Nov. 20, 2014

(54) Device For Tissues Sampling And Grafting

(71) Applicants: Silvano ANDREANI, Borgo Maggiore (SM); Ettore CASALE, Quartu Sant'Elena (CA) (IT); Giorgio MAULLU, Oristano (OR) (IT)

(72) Inventors: Silvano Andreani, Borgo Maggiore (SM); Ettore Casale, Quartu Sant'Elena (IT); Giorgio Maullu, Oristano (IT)

(21) Appl. No.: 14/359,242

(22) PCT Filed: Nov. 22, 2012
(86) PCT No.: PCT/EP2012/073401

§ 371 Date: May 19, 2014

(30) Foreign Application Priority Data
Nov. 23, 2011 (IT) BO2011A 000667

(51) Int. Cl. A61B 17/322; A61B 17/3205

(52) U.S. Cl. CPC A61B 17/322; A61B 17/32053; A61B 2017/320064
USPC 606/132; 606/133

(57) ABSTRACT

A device for tissue sampling and grafting has a handle (2), to whose end is fixed a coupling (4), a needle (6) fixed to the coupling. The device also includes a piston (3) connected to a pin (7). The piston and pin translate between an external condition (A), wherein a tip (73) of the in protrudes beyond the needle, and an internal condition (B), wherein the tip is retracted inside the needle. The device also includes a tip (5), having a truncated free end (51) that can be sharpened, constrained in a roto-translating way to the coupling, placed co-axially and externally to the needle and moved between a retracted condition (C) and an advanced condition.

### TECHNICAL FIELD

The present invention relates to the technical field of surgical devices and particularly relates to a device for tissues sampling and grafting.

In the field of tissue sampling and grafting and especially of those that constitute the follicular units are generally used two specific tools to realize the individual sampling and grafting steps.

The sampling step is performed using an extractor device, similar to a coring drill, consisting of a hole saw, rotated by a micro-motor and connected to a suction unit. Then the hole saw performs a sort of coring of the follicular unit with a part of the surrounding skin tissue which is conveyed by means of a suction unit, generally consisting of a vacuum pump connected through a duct to the device, into a collection container.

The operator performs a series of sampling at the end of which empties the collecting container and evaluates the follicular units suitable for subsequent grafting step.

First the grafting step involves the creation of a series of grafting holes using a drill, then a suction step of a single follicular unit through a needle to be grafted and finally the grafting of this unit within the corresponding follicular grafting hole.

### TECHNICAL PROBLEM

The main disadvantage of the prior art concerns the large number of operations to be performed by the operator to sample and subsequently to graft each follicular unit. Due to the large number of manipulations suffered by each follicular unit, the latter is particularly stressed and “mishandled” so as to significantly reduce the grafting success rate.

Further disadvantage of the prior art concerns the high bleeding of the affected tissues during the grafting step and especially during the graft holes preparation step.

### TECHNICAL SOLUTION

The main object of the present invention is to propose a device for tissue sampling and grafting that reduces the number of operations to be performed also reducing the stress applied to the concerned tissue to be sampled and grafted thereby improving their engraftment chances.

Further object is to simplify and speed up the sampling and grafting operations using a single device.

Another object is to propose a device suitable to the auto transplant operations of epithelial tissues, removing of scars, and epidemic damage due to burns and/or tattoos.

Further object is to propose a device which allows to visually evaluate the taken tissue sample condition before the grafting of the same to ensure a higher grafting success rate.

### BRIEF DESCRIPTION OF DRAWINGS

The characteristics of the invention are highlighted in the following with particular reference to the accompanying drawings wherein:

FIG. 1 shows a front view of a device according to the present invention;

FIGS. 2 and 3 show a front view of the device of FIG. 1 where in some portions have been removed to better illustrate others;

FIGS. 4, 5, 6, 8 to 13 show a front view of respective constituent parts of the device of FIG. 1;

FIG. 7 shows a sectional view of the detail shown in FIG. 6;

FIGS. 14 and 16 respectively show the device wherein some portions have been removed to better show others and its own particular in correspondence of a first operating condition;

FIGS. 15 and 17 respectively show the device wherein some portions have been removed to better show others and its own particular in correspondence of a second operating condition;

FIGS. 18 and 20 respectively show the device wherein some portions have been removed to better show others and its own particular in correspondence of a third operating condition;

FIGS. 19 and 21 respectively show the device wherein some portions have been removed to better show others and its own particular in correspondence of a fourth operating condition;

FIG. 22 shows a top view of a first variant of the device of FIG. 1 wherein some portions have removed to better show others;

FIGS. 23 and 24 show each a front view in median section of the device of FIG. 22 in two operating conditions;

FIG. 25 shows a front view of a second variant of the device of FIG. 1.

### BEST MODE FOR CARRYING OUT THE INVENTION

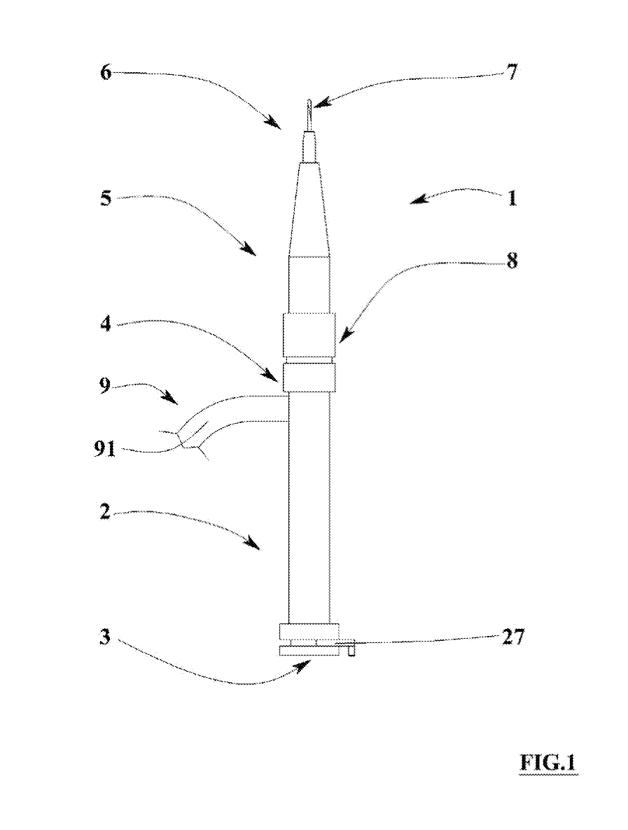

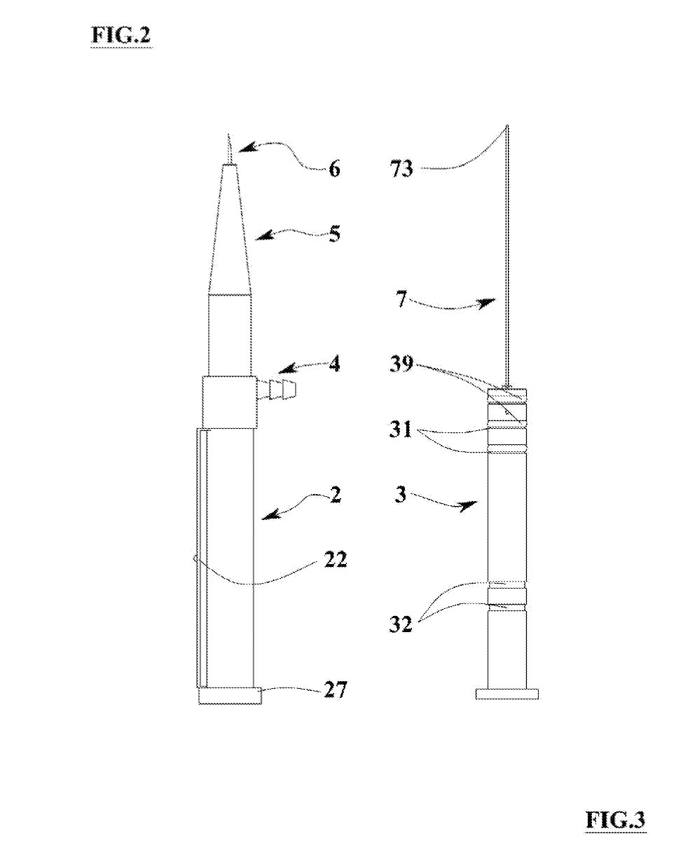

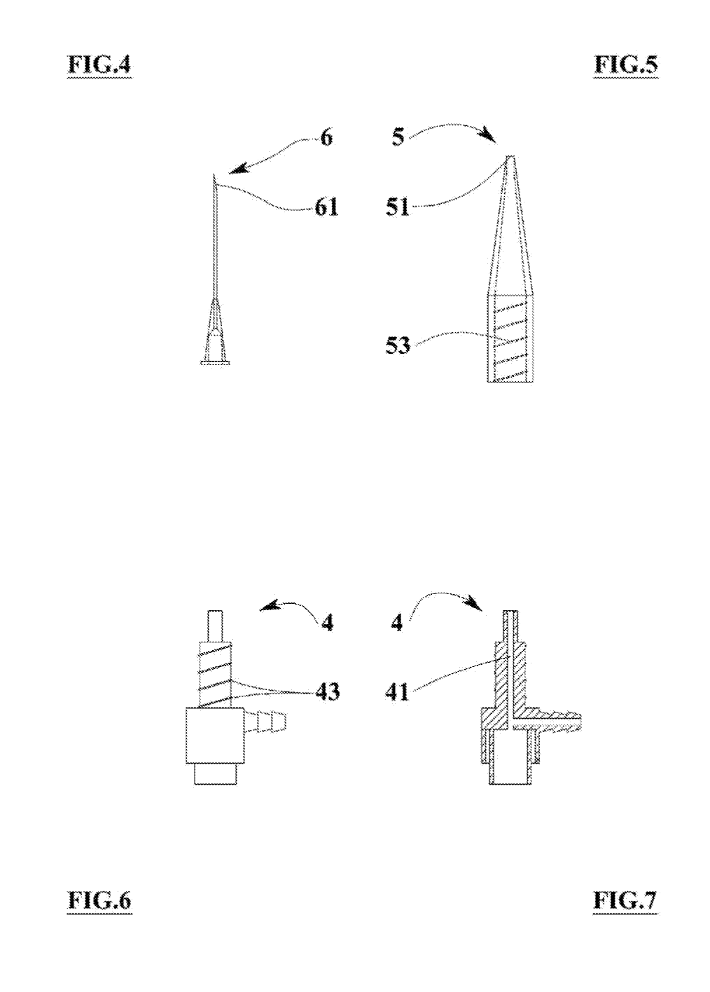

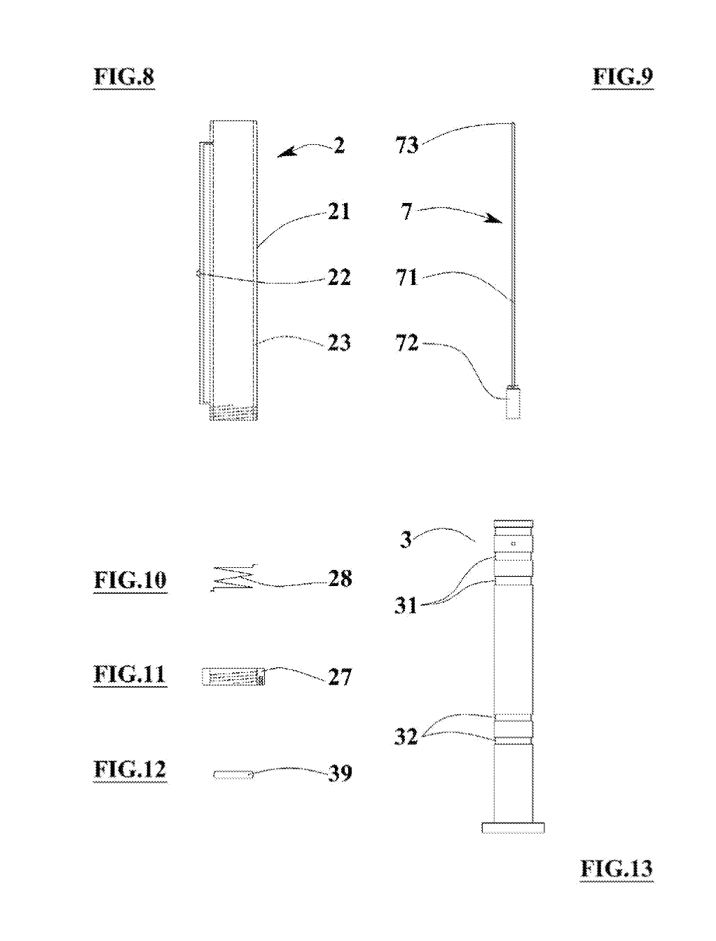

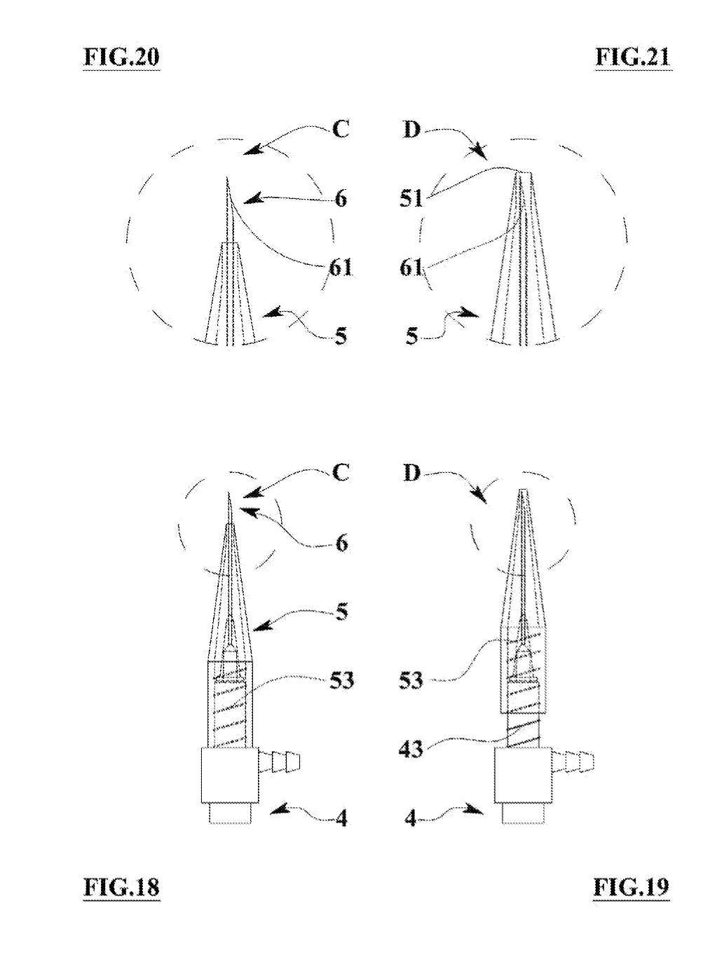

In FIGS. 1 to 25 the number 1 indicates a device for tissues sampling and grafting, such as follicular units, in the operations of hair or skin portions transplant.

In the preferred embodiment, shown in FIGS. 1 to 21, the device 1 comprises a handle means 2 that is elongated shaped and provided with a first longitudinal through hollow 23.

A coupling means 4 is positioned at an end of the handle means 2 and it is removably coupled to the latter. The coupling means 4 has an inner through duct 41 coaxial with the first through hollow 23 and connected thereto.

A needle means 6 equipped with a lanceolate tip 61 is fixed to the coupling means 4 at the opposite end to which the handle means 2 is connected. The needle means 6 is coaxial with the through duct 41.

The diameter of the needle means 6 is chosen according to the type of operation to be performed. Preferred diameters are usually of 0.6-0.8-1.0 mm for the sampling and grafting operations of single or multiple follicular units, while diameters of 1.2-1.5 mm are preferred for sampling and/or grafting for histological, dermal-epidermal, scarring or similar tissue. The person skilled in the art can easily choose the needle means 6 diameter which best suits the specific operation, choosing among the above mentioned or any other diameters, without departing from the scope of the present invention.

Inside the handle means 2 is placed a piston means 3 which slides axially inside the first through hollow 23 and which is equipped with a pin means 7 which is provided with a tip 73 and is fixed to an end of the piston means 3 which remains inside the first through hollow 23.

The pin means 7 slides, together with the piston means 3, inside the through duct 41 and inside the needle means 6. The pin means 7 comprises a needle element 71, having an outer diameter smaller than the inner diameter of the needle means 6, equipped with a coupling element 72 for the removable fixing of the same to the piston means 3.

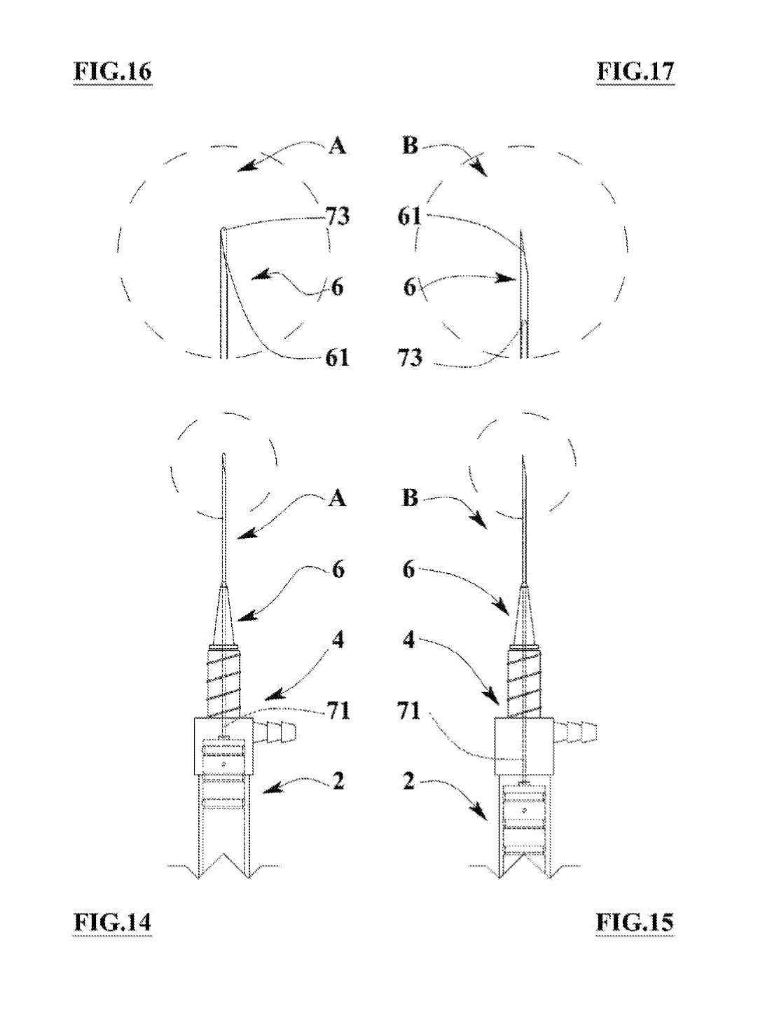

The piston 3 and pin 7 means translate between an external condition A, shown in FIGS. 14 and 16, wherein the tip 73 of the pin means 7 projects beyond the lanceolate tip 61, and an internal condition B, shown in FIGS. 15 and 17, wherein the tip 73 is retracted inside the needle means 6 leaving free an inner portion of the latter.

The piston means 3 presents some sea ling elements 39, such as O-rings, housed in first circumferential grooves 31 that are on the same piston means 3 and parallel to one another. The sealing elements 39 limit the air flow between the handle means 2 and the piston means 3 during the movement of the latter with respect to the handle means, thereby creating an air volume inside the handle means 2 which works as an elastic element opposing to the movement of the piston means 3 with respect to its initial condition wherein it is almost fully inserted in the handle means 2.

The number and shape of the sealing elements 39 are chosen according to the response of the “elastic element” preferred by the operator. A large number of sealing elements 39 provides a slower response, because of the friction generated by the sealing elements 39 opposing to the piston means 3 movement. On the other side, using a limited number of sealing elements 39, up to the limit case of a single sealing element 39, provides a faster response of the “elastic element”. In addition to varying the number of sealing elements 39 is also possible to modify the shape of the same and of their respective first circumferential grooves 31 by varying the free space, or clear span, present among the above mentioned elements, since a larger clear span allows a piston means 3 faster actuation.

The device 1 is connected to a suction means 9 via a first suction duct 91 connected to the coupling means 4, and a thought a flow passage and the duct 41 present in the latter, is in flow communication to the first through hollow 23 and to the needle means 6.

A tip means 5, elongated and tapered near a truncated free end 51 is coupled in a roto-translating way to the coupling means 4 and co-axially surrounds the needle means 6. The tip means 5 moves between a retracted condition C, wherein the lanceolate tip 61 of the needle means 6 protrudes beyond the truncated free end 51 of the tip means 5, and an advanced condition D, wherein the lanceolate tip 61 is completely inside the tip means 5.

The truncated free end 51 has very thin sidewall, as shown in FIGS. 20 and 21, or is sharpened in order to cut the tissue to be sampled.

The roto-translating constraint between the tip means 5 and the coupling means 4 is allowed by the presence of a first threaded element 53, realized inside the tip means 5, to mate a corresponding second threaded element 43 realized on the outer wall of the coupling means 4. The choice of the pitch of the first 53 and second 43 threaded elements is made depending on the desired ratio between the rotation of the tip means 5 with respect to the coupling means 4 and their mutual translation. To obtain a high translation against a limited rotation a coarse-pitch threaded element is used. The person skilled in the art can suitably choose the pitch of the threaded elements without departing from the scope of the present invention.

The handle means 2 comprises also a lock means 27, constituted of an element rotably coupled to the handle means 2, detachably engaging second circumferential grooves 32 present on the piston means 3 to lock the latter 3 in the internal condition B or in intermediate configurations between the external A and internal B ones.

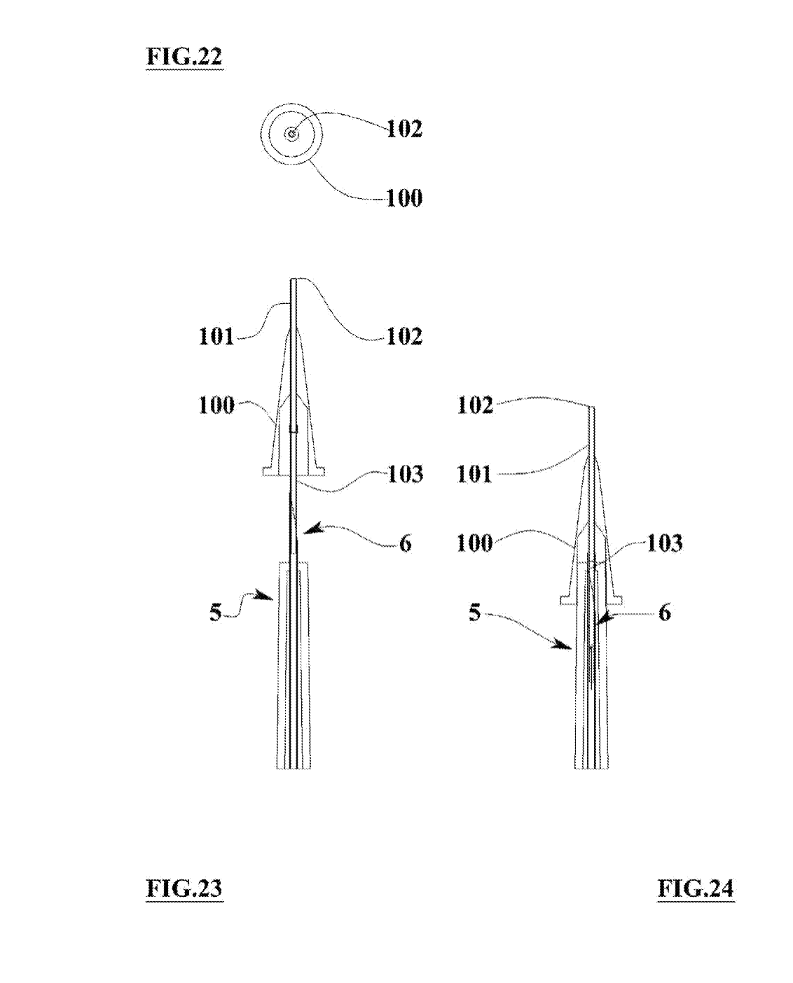

In a first variant of the preferred embodiment, shown in FIGS. 22 to 24, the device 1 also comprises a fixing element 100, for example shaped as a Luer Lock or bayonet or quick coupling or truncated cone, which is removably constrainable to the tip means 5.

The fixing element 100 supports a tubular element 101 in coaxial way with respect to the needle means 6 to which it is in flow communication through a tubular portion 103 having an outer diameter smaller than the inner diameter of the needle means 6 so as to be able to translate at least partially inside the latter 6.

The free end 102 of the tubular element 101 is sharpened and cut along a plane orthogonal to the direction along which the tubular element 101 develops, so as to define a “hole saw”.

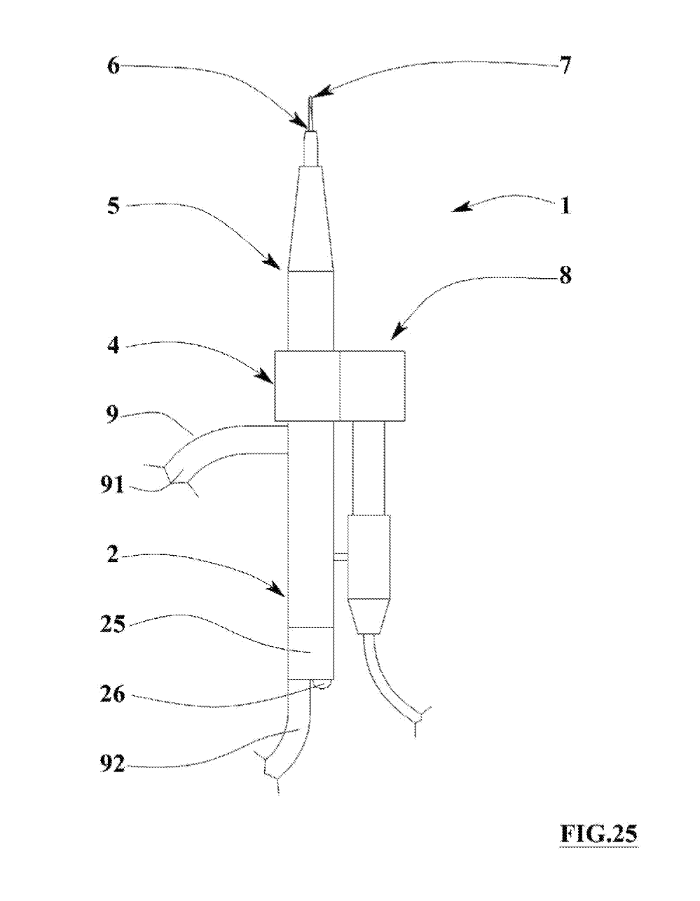

In a second variant of the preferred embodiment, shown in FIG. 25, the device 1 is further equipped with a first actuator means 8 coupled to the coupling means 4 and assigned to rotate the tip means 5 with respect to the coupling means 4. Further in this variant the free end 51 of the tip means 5 can be sharpened. The combination of the first actuator means 8 and the free end 51, that can be sharpened, allows the device to work as a coring drill.

In a third variant of the preferred embodiment, the needle means 6 rotates with respect to the handle means 2 actuated by a second actuator means, such as a torsion spring 28 whose one end is coupled to the handle means 2 and the other end is coupled to the needle means 6.

In a fourth variant of the preferred embodiment, not shown, the device 1 comprises a third actuator means assigned to move the piston means 3 with respect to the handle means 2, and the third actuator means is constituted by a second suction duct 92, connected to the suction means 9 and controlled via a switch element 26, or is constituted by a micro-motor.

In a fifth variant of the preferred embodiment, the device 1 comprises a common switch element 22 coupled to the handle means 2 and assigned to control at the same time the rotation of the needle means 6 and the translation of the piston means 3.

In a sixth variant of the preferred embodiment, the first 8, second 25 and third 28 actuator means are coaxial to the handle means 2, are provided of a hollow driving shaft and are constituted by an actuator chosen among micro-motors, pneumatic, hydraulic or ultrasonic actuators.

In a seventh variant of the preferred embodiment, the device 1 further comprises a display means of the taken tissue sample. This display means is constituted of a transparent portion shaped as a magnifying glass integrated in the tip means 5 near a stop portion wherein the taken tissue sample is retained to visually evaluate the conditions of the sample itself.

Alternatively both the tip means 5 and the needle means 6 are made of transparent materials to allow viewing of the taken tissue sample.

The operation of the device 1 provides that the operator places the device 1 over the portion of tissue to be taken, and in the case of follicular units sampling the device 1 is aligned to the follicular unit same.

In the advanced condition D the device 1 is leaning against the tissue to be taken and the first actuator means is actuated 8 which, by rotating the end 51 of the tip means 5, performs a coring of the affected area around the unit follicular.

Then the piston means 3 is shifted from the external condition A to the internal B one and simultaneously the follicular unit is sucked inside the needle means 6, in the space left by the pin means 7 which is moved integral with the piston means 3.

Then the device 1 is removed from the sampling zone and placed at the grafting area in the retracted condition C, in such a way that the needle means 6 protrudes beyond the free end 51 of the tip means 5.

The end of the needle means 6 is leant on the tissue where the previously taken tissue should be grafted and the needle means 6 rotation is actuated which, via the lanceolate tip 61, creates a circular hole on the affected tissue.

Simultaneously the piston means 3 movement is controlled from the internal condition B to the external A one, and said movement pushes, through the needle means 7, the taken tissue inside the hole created by the lanceolate tip 61.

The simultaneity of operations performed in the grafting step allows to drastically reduce the tissue bleeding, as the graft itself acts as a buffer.

The operation of the first variant of the preferred embodiment provides that the operator constraints the fixing element 100 on the tip means 5, taking care to slide the tubular portion 103 within the needle means 6.

Subsequently, the operator presses the end 102 over the portion of tissue to be sampled and actuates the actuator means which rotates the tip means 5 and consequently also the fixing element 100 and its end 102. In this way the end 102 creates a circular incision around the portion of tissue concerned, which is then sucked inside the needle means 6, in a way similar to the described above operation.

To perform the grafting of the taken tissue, the operator removes the fixing element 100 and operates the steps of the above described operation.

The operation of the seventh variant of the preferred embodiment provides an additional step of visual evaluation of the taken tissue before its grafting by observing the latter through the display means or the transparent portions present on the tip means 5 and the needle means 6.

The main advantage of the present invention is to provide a device for tissue sampling and grafting that reduces the number of operations to be performed also reducing the stress applied to the tissues concerned, to be sampled and grafted, thereby improving their engraftment chances.

Further advantage is to simplify and speed up the sampling and grafting operations by using a single device.

Another advantage is to provide a device suitable to the auto transplant operations of epithelial tissues, removing of scars, epidemic damage due to burns and/or tattoos.

Further advantage is to provide a device that allows to visually evaluate the conditions of the taken tissue sample before grafting the same to ensure a higher grafting success rate.

1-15. (canceled)

16. A device for tissue sampling and grafting comprising:
a handle having an end fixed to a coupling;
a needle having a lanceolate tip at a distal end thereof and a proximal end fixed to the coupling;
the handle having a first through hollow communicating with a longitudinal through duct located inside the coupling and communicating with the needle;
a piston being slidable into the first through hollow and having a pin having a tip, slidable inside the longitudinal through duct within the needle, said piston and pin adapted to translate between an external condition in which the tip protrudes beyond the lanceolate tip, and an internal condition, in which the tip is retracted inside the needle;
a suction means communicating with the first through hollow and with the longitudinal through duct;
a truncated tip which is sharpenable being placed co-axially outside of the needle and being roto-translatable with respect to the coupling between a retracted condition, in which the lanceolate tip protrudes beyond the truncated tip, and an advanced condition, in which the lanceolate tip is completely inside the truncated tip.

17. The device according to claim 16 wherein the handle has a lock which engages at least one of a set of second circumferential grooves located on the piston to lock the piston in the internal condition or in an intermediate condition between the external condition and the internal condition.

18. The device according to claim 16 wherein the truncated tip has at least one first inner threaded element engaging at least one second threaded element provided on an outside of the coupling for rote-translating the truncated tip relative to the coupling.

19. The device according to claim 16 further comprising a tip actuator, connected to the coupling, for rotating the truncated tip with respect to the coupling.

20. The device according to claim 19 wherein the truncated tip, rotated by the tip actuator, performs as a coring drill.

21. The device according to claim 16 further comprising a needle actuator which rotates the needle with respect to the handle.

22. The device according to claim 21 wherein the needle actuator consists of a torsion spring having an end constrained to the handle.

23. The device according to claim 16 further comprising a piston actuator, constrained to the handle, for translating the piston, the piston actuator being a micro-electric, a mechanical, an hydraulic or an ultrasonic actuator, or being operable via suction.

24. The device according to claim 16 further comprising a needle actuator which rotates the needle with respect to the handle, a piston actuator, constrained to the handle, for translating the piston, and, a switch element constrained to the handle for controlling simultaneously the needle rotation and the piston translation.

25. The device according to claim 16 further comprising a tip actuator, connected to the coupling, for rotating the truncated tip with respect to the coupling, a needle actuator which rotates the needle with respect to the handle, and a piston actuator, constrained to the handle, for translating the piston, at least one of the tip actuator, needle actuator and piston actuator is coaxial with the handle, is equipped with a hollow motor shaft and is a micro-electric, a pneumatic, an hydraulic or an ultrasonic actuator.

26. The device according to claim 16 further comprising a display for displaying a taken tissue sample.

27. The device according to claim 16 wherein the truncated tip and needle are at least partially transparent to allow viewing of a taken tissue sample.

28. The device according to claim 27 wherein the truncated tip has a transparent portion shaped as a magnifying glass, near a stop portion thereof wherein a taken tissue sample is retained for visually evaluating a condition of the taken tissue sample.

29. The device according to claim 16 further comprising a fixing element detachably constrainable to the truncated tip for supporting a tubular element provided with an end sharpened on an orthogonal geometric plane with respect to the tubular element, and being coaxial to the needle with which the tubular element is in flow communication.

30. The device according to claim 29 wherein the tubular element is in flow communication with the needle via a tubular portion thereof that has an outer diameter smaller than an inner diameter of the needle such that tubular portion is at least partially translatable therein.
